(19) United States
(12) Patent Application Publication
(10) Pub. No.: US 2017/0157044 A1
(43) Pub. Date: Jun. 8, 2017

(54) DRY POWDER MIXING PROCESS

(71) Applicants: Alfred E. Tiefenbacher GmbH & Co. KG, Hamburg (DE); Nanopharm Ltd., Bath (GB)

(72) Inventors: Claudia Meyer, Hamburg (DE); Robert Price, Chepstow (GB); Jagdeep Shur, Bath (GB)

(73) Assignees: Alfred E. Tiefenbacher GmbH & Co. KG, Hamburg (DE); Nanopharm Ltd., Bath (GB)

(21) Appl. No.: 15/363,317

(22) Filed: Nov. 29, 2016

(30) Foreign Application Priority Data
Dec. 3, 2015 (EP) 15 197 874.9

(51) Int. Cl. A61K 9/00; A61K 31/439; A61K 9/14

(52) U.S. Cl. CPC A61K 9/0075; A61K 9/145; A61K 31/439

(57) ABSTRACT

The present invention relates to a simple, robust and effective process for preparing a dry powder inhalation formulation containing at least one drug, preferably a highly active drug, more preferably Tiotropium bromide or Tiotropium bromide monohydrate, and a lactose carrier. The process is characterized in that a three-layered composition containing a drug layer in between layers of the lactose carrier is mixed and the obtained preblend is mixed with additional lactose carrier. The process combines a simple manufacturing procedure with an excellent content uniformity obtained over a wide range of mixing parameters.

### CROSS-REFERENCE TO RELATED APPLICATIONS

The present application claims priority from European Patent Application No. 15 197 874.9 filed Dec. 3, 2015, the disclosure of which is hereby incorporated herein by reference.

### BACKGROUND OF THE INVENTION

The present invention relates to a process for preparing a dry powder formulation suitable for inhalation containing at least one drug and a lactose carrier.

Powder formulations for inhalation are typically administered by means of dry powder inhalers (DPIs). Several types of DPI devices exist such as reservoir devices, capsule devices and blister devices. In a typical device, the powder constitutes of a relatively large amount of excipient with a small amount of drug, typically in the order of 1-2% of the loading. In order to deposit the drug particles in the deep lungs, the mass median aerodynamic diameter (MMAD) of the drug particles is 10 μm or less, typically in the range of 0.5-5 μm.

The larger excipient particles are responsible for the flow properties of the dry powder inhalation formulation when portioning the inhalation powder in single doses, e.g. when filling into capsules or upon loading a dose into the reservoir of the inhalation device, because due to the low amount of drug powder bulk properties, such as powder flow, are expected to rely largely on the excipient properties. Moreover, powder bulk properties also play a major role in the inhalation process. Dry powder inhalation formulations are typically prepared by blending the drug and the excipient. During the mixing process, the drug particles are adhered to the excipient particles. The excipient particles thus function as carrier particles, and it is important that the drug may be detached from the carrier particles during inhalation. Therefore, on the one hand, the interaction between drug and carrier should be strong, so that the generation of agglomerates of drug particles is prevented. On the other hand, the interaction should be weak enough to be broken during the inhalation event. A suitable balance of these properties is provided by lactose, and various lactose grades are commercially available for the preparation of dry powder inhalation formulations.

Hence, providing good flow characteristics of the dry powder inhalation formulation and an appropriate adherence of the drug to the carrier particles are of great importance. Moreover, the uniformity of the dry powder inhalation formulation is of great importance, too. Content uniformity largely depends on the mixing process used for the preparation of the dry powder inhalation formulation. Particularly, the content uniformity is a crucial factor in highly active drug formulations. Those formulations typically contain a very small proportion of active ingredient to deliver single doses in the microgram range, or even less. Examples of those highly active substances are anticholinergic compounds like Ipratropium bromide or Tiotropium bromide and β-sympathomimetics like Formoterol or Salmeterol.

Flow characteristics, adherence of the drug on the carrier particles and content uniformity eventually serve the purpose of ensuring dosing accuracy. Various techniques are known in the art for providing above characteristics of the dry powder inhalation formulation.

WO 93/11746 reports that coarse excipient particles provide good flow characteristics but negatively influence the liberation of the drug in the lungs. In order to overcome this problem a mixture of coarse excipient particles having an average particle size of 20 μm or more and fine excipient particles having an average particle size of 10 μm or less is used.

According to DE 44 25 255 a high bulk density of the dry powder inhalation formulation and similar bulk and tapped densities are decisive factors for achieving good flow characteristics. It was found that mixing a micronized drug with excipient particles having an average particle size in the range of 200 μm to 1,000 μm, preferably, 300 μm to 600 μm, provides spherical carrier particles with the drug deposited on the surface.

WO 2013/109214 describes a process for preparing a dry powder inhalation formulation containing budesonide. In this process, the main part of the coarse excipient is divided into four equal fractions, and the remaining part of the coarse excipient is mixed with the fine excipient to obtain a fine excipient/coarse excipient mixture. Budesonide is mixed with one fraction of coarse excipient to obtain a budesonide/coarse excipient mixture. The dry powder inhalation formulation is then prepared as follows. Two fractions of coarse excipient are placed in a blender. Subsequently, the budesonide/coarse excipient mixture, the fine excipient/coarse excipient mixture and finally the fourth fraction of coarse excipient are added. Blending the four-layered composition gives the final dry powder inhalation formulation.

According to the process described in WO 2004/017918 a first portion of the drug is mixed with an excipient to obtain a first preblend that is subsequently mixed with a second drug to form a second preblend. Finally, the second preblend is mixed with the remaining portion of the first drug to obtain the dry powder inhalation formulation.

WO 2007/068443 describes a process for the preparation of a dry powder inhalation formulation, in which a drug is mixed with a force-controlling agent, e.g. a phospholipids, titanium dioxide, aluminium dioxide, silicon dioxide, starch or salts or fatty acids, in order to obtain drug particles having the force-controlling agent deposited on the surface. This composite material is subsequently mixed with a small portion of the carrier material to obtain a preblend that is subsequently mixed with the remaining portion of the carrier material. The force-controlling agent serves the purpose to adjust the interaction between drug and carrier.

EP 1 326 585 discloses a mixing process, in which the drug and the excipient are divided into substantially equal fractions. These fractions are placed in alternate layers in a suitable mixer, whereby the excipient is placed in the mixer first. A relatively high content uniformity is obtained if at least 10, preferably 20, and even more preferably more than 30 layers are put together in the mixer to form the composition.

Hence, various methods have been suggested in the art to prepare a dry powder inhalation formulation showing good flow characteristics and content uniformity. These methods may be classified as dilution methods, in which a drug/carrier preblend is subsequently diluted with additional carrier, or as multiple-layer mixing processes, in which the entire drug and the entire excipient are placed in a mixer as alternate, equal layers and subsequently blended.

They suffer, however, from several setbacks, as shown in example 1 of EP 1 326 585. In a first step, 33.5 kg of a lactose preblend was prepared by 31 and 30 layers of coarse and fine lactose, respectively, which were subsequently mixed in a free fall mixer. In a second step, a total of 32.87 kg of the lactose preblend was then mixed with 0.13 kg micronized Tiotropium bromide monohydrate. The excipient was thereby added in 46 layers, the active ingredient in 45 layers, and subsequently mixed in a free fall mixer, to ensure a proper content uniformity of the mixture.

It can easily be recognized that a mixing process requiring a total of 152 weighting steps is extremely unfavourable. Furthermore, the very small amount of the single Tiotropium bromide monohydrate layer (0.003 kg) compared to the overall scale of the preparation will cause additional handling problems.

It was an objective of the present invention to provide a process for preparing a dry powder inhalation formulation showing excellent flow characteristics and content uniformity by a simple, robust and effective process.

This objective is attained by the subject matter as defined in the claims.

### BRIEF SUMMARY OF THE INVENTION

The present invention relates to a process for preparing a dry powder inhalation formulation containing at least one drug and a lactose carrier, comprising the steps:
a) splitting the lactose carrier into a first, a second and a third portion,
b) mixing the first and the second portion of the lactose carrier and the drug by
   i) placing the first portion of the lactose carrier in a mixer to obtain a first layer,
   ii) placing the entire drug on top of the first lactose layer to obtain a second layer,
   iii) placing the second portion of the lactose carrier on top of the second layer to obtain a third layer,
   iv) blending the three-layered composition obtained in step (iii) to obtain a preblend,
   v) optionally sifting the preblend,
c) mixing the preblend obtained in step (b) and the third portion of the lactose carrier to obtain the dry powder inhalation formulation, and
d) optionally sifting the dry powder inhalation formulation.

The particle size of the lactose carrier used in the dry powder inhalation formulation must be suitable to yield good flowability. The particle size is typically determined by laser diffraction (Sympatec or Malvern). Various grades of lactose suitable for inhalation are commercially available (e.g. Lactohale®, DFE, Pharma).

According to a preferred embodiment of the present invention, the lactose carrier is prepared by mixing coarse lactose and fine lactose. While coarse lactose improves the flowability of the dry powder inhalation formulation, fine lactose (including micronized lactose) allows uniform attachment of the one or more drugs to the carrier particles. Coarse lactose has a volume median diameter (VMD) typically in the range of 50-150 μm, while the VMD of fine lactose is typically below 50 μm, usually in the range of 15-30 μm. Micronized lactose has a VMD of 10 μm or less, typically 5 μm or less. Preferably, the fine lactose portion of the lactose carrier is 30% by weight or less, more preferred 10-30% by weight and most preferred 20-30% by weight.

The following drugs in particular can be employed in the dry powder inhalation formulation of the present invention: terbutaline, salbutamol, salmeterol, formoterol, budesonide, ciclesonide, mometasone, fluticasone, beclometasone, ipratropium and tiotropium, as well as salts and ester prodrugs thereof. The process of the present invention is in particular suitable for preparing a dry powder inhalation formulation containing highly active drugs, preferably Tiotropium bromide, and even more preferred Tiotropium bromide monohydrate.

It is preferred that the lactose carrier is prepared in the same way as the dry powder inhalation formulation, i.e. by a process comprising the steps:
a1) splitting the coarse lactose into a first, a second and a third fraction,
a2) placing the first fraction of the coarse lactose in a mixer to obtain a first layer,
a3) placing the entire fine lactose on top of the first layer to obtain a second layer,
a4) placing the second fraction of the coarse lactose on top of the second layer to obtain a third layer,
a5) blending the three-layered composition obtained in step (a4) to obtain a preblend,
a6) optionally sifting the preblend,
a7) mixing the preblend obtained in step (a5) or (a6) and the third fraction of the coarse lactose to obtain the lactose carrier, and
a8) optionally sifting the lactose carrier.

Preferably, the first, second and third fractions of the coarse lactose in the preparation of the lactose carrier are equal.

### BRIEF DESCRIPTION OF THE DRAWINGS

FIG. 1 displays an aspect of the invention as a flow diagram.

FIG. 2 displays an aspect of the invention as a flow diagram.

### DETAILED DESCRIPTION

According to a preferred embodiment of the present invention, the amount of the first portion, the second portion and the third portion of the lactose carrier in the preparation of the dry powder inhalation formulation each is at least 10% by weight, based on the total weight of the lactose carrier. It is even more preferred that the amounts of the respective portions of the lactose carrier are not equal. It has been found that excellent content uniformity was achieved if the weight ratio first portion/second portion/third portion of the lactose carrier is 7/2/1, 5/1/4, 5/2/3, 4/1/5, or 2/1/7. Preferably, the mixing device for preparing the lactose carrier as well as the dry powder inhalation formulation of the present invention is a Turbula® mixer.

According to a preferred embodiment of the present invention, the dry powder inhalation formulation consists of tiotropium bromide, preferably tiotropium bromide monohydrate, and the lactose carrier, preferably lactose monohydrate.

The following examples are intended to further illustrate the present invention.

### Examples

Content uniformity as expressed by the target content per dosage unit (% Assay), the relative standard deviation (% RSD) and the acceptance value (AV) is determined according to USP 38 <905>.

### Example 1. Manufacture of Lactose Carrier

### 1. Pre-Sieving

1.1. Pre-sieve fine lactose through a 250 μm sieve.

1.2. Pre-sieve coarse lactose through a 250 μm sieve.

### 2. Lactose Carrier Manufacture

Add ⅓ of the coarse lactose, add all fine lactose, add approximately ⅓ of the coarse lactose.
Seal the mixing jar and mix on the Turbula mixer at 22 rpm for 15 minutes.
Pass the mixture through 250 μm sieve
Add remaining ⅓ of the coarse lactose.
Seal the mixing jar and mix on the Turbula mixer at 22 rpm for 15 minutes.
Once blending is completed, sieve the lactose carrier through 250 μm sieve and collect.

The fine lactose content of the lactose carrier is 25% by weight.

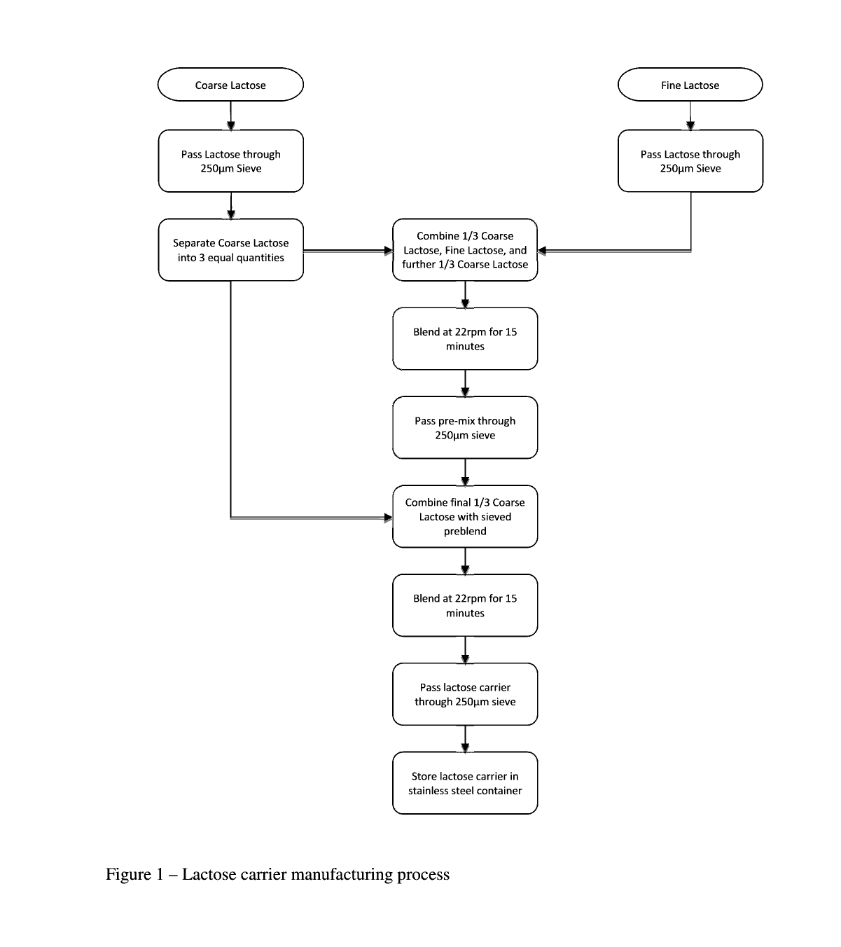

FIG. 1 displays this lactose carrier manufacturing process as a flow diagram.

### Examples 2-6 Preparation of Dry Powder Inhalation Formulation Containing Tiotropium Bromide Monohydrate (Nominal Concentration 0.41% w/w Active Ingredient)

### 1. Pre-Sieving

1.1. Pre-sieve lactose carrier through a 250 μm sieve.
1.2. Pre-sieve tiotropium bromide monohydrate through a 250 μm sieve.

### 2. Layer Preparation

2.1. Separate the lactose carrier into 10 equal quantities.

### 3. Formulation Construction

3.1. Add 5 quantities of lactose carrier into the blending vessel.
3.2. Add the whole amount of tiotropium bromide monohydrate into the blending vessel directly on top of the lactose.
3.3. Add 2 quantities of lactose carrier into the vessel directly on top of the tiotropium bromide monohydrate.
3.4. Seal the vessel and place into the Turbula mixer at 22 rpm for 15 minutes.
3.5. Add final 3 quantities of lactose carrier on top of the blend.
3.6. Reseal the vessel, place into the Turbula mixer at 22 rpm for further 15 minutes.
3.7. Remove the vessel and pass the formulation through a 250 μm sieve.

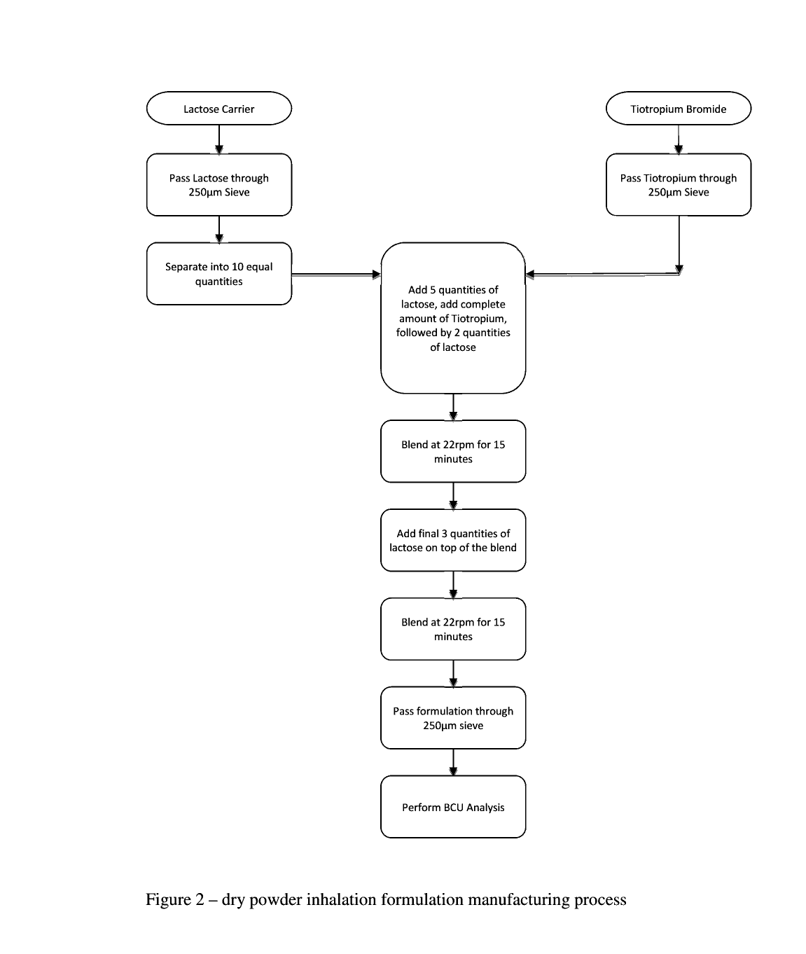

FIG. 2 displays this dry powder inhalation formulation manufacturing process as a flow diagram.

Above process was repeated with the following weight ratios first portion/second portion/third portion of the lactose carrier: 7/2/1, 5/1/4, 4/1/5 and 2/1/7.

TABLE 1
Weight ratio of first portion/second portion/third portion of the
lactose carrier

| Ex. | 1^{st} Blending (step b) | | 2^{nd} Blending (step c) |
|---|---|---|---|
| 2 | 5 Parts Lactose Carrier | 2 Parts Lactose | 3 Part Lactose Carrier |
| | | Carrier | |
| 3 | 7 Parts Lactose Carrier | 2 Parts Lactose | 1 Part Lactose Carrier |
| | | Carrier | |
| 4 | 5 Parts Lactose Carrier | 1 Part Lactose | 4 Parts Lactose Carrier |
| | | Carrier | |
| 5 | 4 Parts Lactose Carrier | 1 Part Lactose | 5 Parts Lactose Carrier |
| | | Carrier | |
| 6 | 2 Parts Lactose Carrier | 1 Part Lactose | 7 Parts Lactose Carrier |
| | | Carrier | |

TABLE 2
Content uniformity

| Ex. | % Assay | % RSD | AV |
|---|---|---|---|
| 2 | 99.9 | 3.3 | 7.8 |
| 3 | 100.4 | 2.9 | 7.1 |
| 4 | 102.2 | 4.8 | 12.5 |
| 5 | 101.0 | 3.2 | 7.7 |
| 6 | 101.5 | 4.1 | 10.0 |

1. A process for preparing a dry powder inhalation formulation containing at least one drug and a lactose carrier, comprising the steps:
a) splitting the lactose carrier into a first, a second and a third portion,
b) mixing the first and the second portion of the lactose carrier and the drug by
i) placing the first portion of the lactose carrier in a mixer to obtain a first layer,
ii) placing the entire drug on top of the first lactose layer to obtain a second layer,
iii) placing the second portion of the lactose carrier on top of the second layer to obtain a third layer,
iv) blending the three-layered composition obtained in step (iii) to obtain a preblend,
v) optionally sifting the preblend,
c) mixing the preblend obtained in step (b) and the third portion of the lactose carrier to obtain the dry powder inhalation formulation, and
d) optionally sifting the dry powder inhalation formulation.

2. The process according to claim 1, wherein the lactose carrier is prepared by mixing coarse lactose and fine lactose.

3. The process according to claim 2, comprising the steps:
a1) splitting the coarse lactose into a first, a second and a third fraction,
a2) placing the first fraction of the coarse lactose in a mixer to obtain a first layer,
a3) placing the entire fine lactose on top of the first layer to obtain a second layer,
a4) placing the second fraction of the coarse lactose on top of the second layer to obtain a third layer,
a5) blending the three-layered composition obtained in step (a4) to obtain a preblend,
a6) optionally sifting the preblend,
a7) mixing the preblend obtained in step (a5) or (a6) and the third fraction of the coarse lactose to obtain the lactose carrier, and
a8) optionally sifting the lactose carrier.

4. The process according to claim 3, wherein the first, second and third fractions of the coarse lactose are equal.

5. The process according to claim 2, wherein the fine lactose content of the lactose carrier is 30% by weight or less, preferably 10-30% by weight, more preferred 20-30% by weight.

6. The process according to claim 1, wherein the amount of the first portion, the second portion and the third portion of the lactose carrier each is at least 10% by weight, based on the total weight of the lactose carrier.

7. The process according to claim 6, wherein the amount of the first portion of the lactose carrier is 10-70% by weight, the amount of the second portion of the lactose carrier is 10-20% by weight, and the amount of the third portion of the lactose carrier is 10-70% by weight, based on the total weight of the lactose carrier.

8. The process according to claim 6, wherein the weight ratio of the first portion/second portion/third portion of the lactose carrier is 7/2/1, 5/2/3, 5/1/4, 4/1/5 or 2/1/7.

9. The process according to claim 1, wherein the dry powder inhalation formulation consists of tiotropium bromide, preferably tiotropium bromide monohydrate, and the lactose carrier, preferably lactose monohydrate.
